(19) United States
(12) Patent Application Publication
(10) Pub. No.: US 2025/0237658 A1
(43) Pub. Date: Jul. 24, 2025

(54) RAPID MULTIPLEXED ANALYSIS OF RELEASED GLYCANS BY DUAL-STAGE MICROFLUIDIC CAPTURE AND PARALLEL FLUORESCENT TAGGING PRIOR TO LC ANALYSIS

(71) Applicant: Waters Technologies Corporation, Milford, MA (US)

(72) Inventor: Lindsay Morrison, Waltham, MA (US)

(73) Assignee: Waters Technologies Corporation, Milford, MA (US)

(21) Appl. No.: 19/025,097

(22) Filed: Jan. 16, 2025

Related U.S. Application Data
(60) Provisional application No. 63/622,702, filed on Jan. 19, 2024.

(51) Int. Cl. G01N 33/58; G01N 33/577

(52) U.S. Cl. CPC G01N 33/582; G01N 33/577; G01N 2333/4724; G01N 2333/98; G01N 2400/10

(57) ABSTRACT

The present technology provides a description of a system and method for purifying glycans. The system includes a polypeptide capture device fluidically coupled to a glycan capture and labeling device. The polypeptide device includes an affinity capture agent that can capture glycoproteins. The glycan capture and labeling device includes glycan affinity capture agents (e.g., lectin proteins) that can capture specific glycans released from glycoproteins in the polypeptide capture device. The captured glycans are subsequently tagged with a dye. The tagged glycans are removed from the lectins and passed into an analytical device.

### CROSS REFERENCE TO RELATED APPLICATIONS

This application claims priority to and benefit of U.S. provisional application No. 63/622,702, filed Jan. 19, 2024 and entitled “Rapid Multiplexed Analysis of Released Glycans by Dual-Stage Microfluidic Capture and Parallel Fluorescent Tagging Prior to LC Analysis”; the content of which is incorporated herein by reference in its entirety.

### FIELD OF THE TECHNOLOGY

The present disclosure relates to the use of affinity chromatography for the analysis of glycans.

### BACKGROUND

Protein glycosylation can play an important role in defining in vivo bioactivity, stability, solubility, and metabolic fate of biotherapeutics. Glycosylation is a post-translational modification and, unlike transcription, is a non-template-driven enzymatic modification process-its structural status depending largely on production methods and conditions.

Many novel recombinant glycoprotein therapeutics—such as glycoprotein hormones, cytokines, clotting factors, and monoclonal antibodies—are in clinical use. The glycan profile of these products is often an important consideration in the development of biotherapeutics. Glycan profiles are additionally unique in that they have been demonstrated to fluctuate as a function of cell culture media components, temperature, pH, culture time, clonal line, and other variables during the synthesis of the glycoprotein. For novel therapies, the presence of immunogenic glycoforms such as high mannose or afucosylated species can derail a drug candidate in a development pipeline. Contract organizations and biosimilar projects must additionally achieve robustly similar glycoform distributions to match regulatory filings. Glycan characterization, however, can be difficult. Improved and efficient methods of performing glycan characterization is therefore becoming increasingly important.

### SUMMARY

The problems associated with glycan characterization are mitigated by the use of a prefractionation and labeling of the glycans prior to chromatographic analysis. In an embodiment, a method of capturing and labeling glycans released from a protein comprises passing the protein through a polypeptide capture device comprising an affinity capture agent, wherein at least a portion of the protein passing through the polypeptide capture device is captured by the affinity capture agent. The captured protein is treated with a glycanase such that one or more glycans are removed from the captured protein. The released glycans are passed into a glycan capture and labeling device. The glycan capture and labeling device comprises one or more glycan affinity capture agents (e.g., lectin proteins), which have an affinity for a specific structural feature of a glycan. The captured glycans are cleaned and labeled with a dye (e.g., a fluorescent dye).

In a preferred embodiment, the glycan capture and labeling device includes a plurality of wells, each of the wells containing a different kind of glycan affinity capture agent. For example, each well can contain a different class of lectin proteins. When the glycans released from the protein pass over the well, the glycan affinity capture agents in the well selectively bind to one or more types of glycans. In this way the glycans can be prefractionated prior to chromatographic analysis.

In an embodiment, the glycan capture and labeling device includes one or more reagent reservoirs coupled to the one or more of the wells. Each reagent reservoir includes a different dye. During use, the dye is released from each reagent reservoir into a single well of the one or more wells. In this manner, each well receives a unique dye that will only label the glycans that are captured in the well. When a plurality of wells are present, one reagent reservoir is fluidically coupled to each of the wells, with each reagent reservoir containing a different dye. Some embodiments feature two wells, each well having a different glycan affinity capture agent coupled to an interior surface. Certain embodiments feature four or more wells (e.g., six wells), where each well is associated with a distinctive glycan affinity capture agent.

The use of different dyes for each well of the glycan capture and labeling device can provide enhanced detection of the different glycan species. For example, fluorescent dyes used in the wells can have different fluorescence which allows monitoring of different classes of glycans using different wavelengths. Additionally, the dye coupled to the glycan can alter the chromatographic behavior (e.g., the retention time) of the glycans, depending on the analytical method being used to study the glycans. The change in retention time can help separate glycans that typically have similar elution rates.

### BRIEF DESCRIPTION OF THE DRAWINGS

The technology will be more fully understood from the following detailed description taken in conjunction with the accompanying drawings, in which:

FIG. 1A depicts a typical released glycan panel for the NIST monoclonal antibody;

FIG. 1B depicts a typical released glycan panel for an antibody with dual N-linked glycosylation sites.

FIG. 2 depicts a system for isolating and tagging a glycan.

FIG. 3 depicts a top view of a glycan labeling and capture device.

FIG. 4 shows a projection view of a glycan capture and labeling device.

FIG. 5 shows an exploded view of the glycan capture and labeling device.

FIG. 6 shows a chromatogram depicting enrichment of high mannose glycoforms following lectin affinity binding and elution from a retention time of 0 to 40 minutes. FIG. 6 also provides an expanded view of a portion of the chromatogram between 11 and 30 minutes.

FIG. 7 shows a chromatogram depicting unbound “breakthrough” material from the binding step generated during the enrichment of high mannose glycoforms from a retention time of 0 to 40 minutes. FIG. 7 also provides an expanded view of a portion of the chromatogram between 11 and 30 minutes.

FIG. 8A shows a chromatogram of residual dye peaks as separated and measured by HILIC-FLR for the eluted material of FIG. 6.

FIG. 8B shows a chromatogram of residual dye peaks as separated and measured by HILIC-FLR for the breakthrough material of FIG. 7.

FIG. 8C shows the quantitation of dye peaks provided in FIG. 8A and FIG. 8B.

FIG. 9 provides a graph illustrating the results of a kinetics experiment for binding. The graph plots percentage of bound material to on-binding time in minutes and demonstrates that binding is complete within ˜5 minutes. Glycans were bound to the mannose lectin, washed, and then eluted. Species were quantified using a subsequent HILIC-FLR separation.

### DETAILED DESCRIPTION

Glycoproteins are composed of one or more oligosaccharide chains covalently attached to amino acid side-chains of a base protein. The oligosaccharide chains that are attached to the base protein are known as “glycans”.

Typically, glycoproteins are created during the synthesis of glycoprotein therapeutics. Glycan profiles (defined herein as the number and type of glycans attached to the base protein) can be unique to a glycoprotein product. The glycan profile, however, can vary based on the cell culture media components, temperature, pH, culture time, clonal line, and other variables during glycoprotein synthesis.

Glycans are composed of a plurality of carbohydrate base units. The presence and absence of certain carbohydrates in the glycan can create significant differences in the pharmacological properties of the glycoprotein. Table 1 shows the effect of various oligosaccharides present in the glycan on pharmacological properties of the glycoprotein.

TABLE 1

| IgG Glycan | | Classification |
|---|---|---|
| Structure | Clinical Impact | (S/E) |
| Core | Decreases binding to IgG Fc receptor, | Efficacy |
| Fucose (%) | resulting in a decrease in | |
| | antibody-dependent cell-mediated | |
| | cytotoxicity (ADCC) | |
| Terminal | Decreases the complement-dependent | Efficacy |
| Galactose (%) | cytotoxicity (CDC) | |
| High mannose | Can impact clearance rate, or drug | Efficacy, |
| species (%) | half-life (pharmacokinetics) | Safety |
| Sialylated | Higher ratios of α2,6 sialylation can | Efficacy, |
| species (%) | increase ADCC & serum half-life, | Safety |
| | decrease inflammatory response | |
| Galactosylation | Can increase complement-dependent | Efficacy, |
| (%) | cytotoxicity (CDC); has been | Safety |
| | implicated in autoimmune diseases; | |
| | modulates inflammatory response | |

A typical procedure for glycan characterization requires purification of the target glycoprotein, enzymatic release of the glycans, buffer exchange, the addition of a fluorescent mass tag, and finally analysis by liquid chromatography coupled with both fluorescence and mass detection. This assay, while extremely accurate and powerful, requires significant sample preparation and lengthy chromatographic runs of 1 hour or more. Faster chromatography has been achieved using only mass detection; however, this approach trades less accuracy in the quantitation for analysis time and suffers from robustness issues.

The standard approach for managing the need for glycan data across a large design of experiments is to only perform the assay on the top 3-5 candidates from each round of parallel bioreactor experiments or to limit the collection of the data to a few days of sampling. Such an approach not only risks selection of less optimal candidates in terms of product quality, but also restricts the use of temporal parameterization of feed, temperature, pH, and dissolved oxygen.

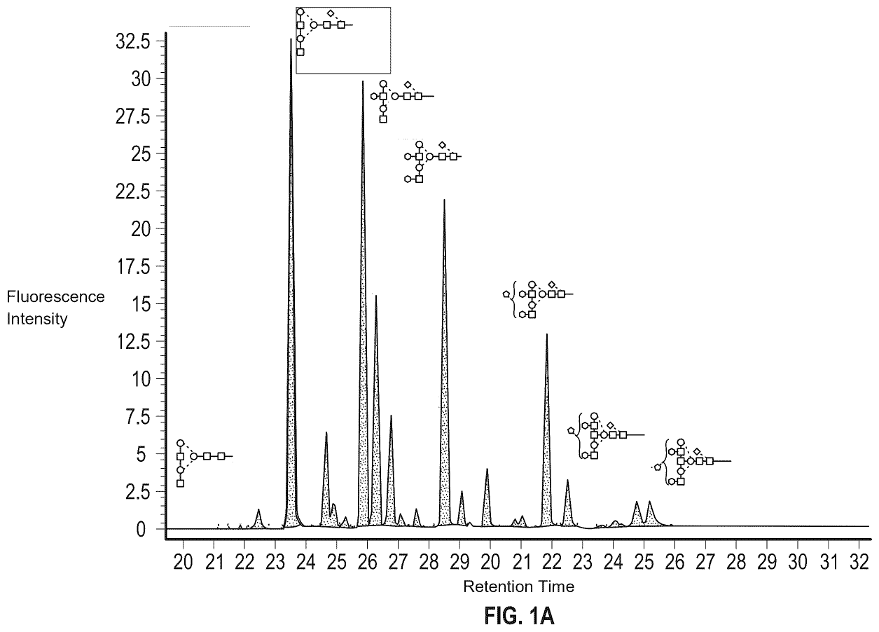

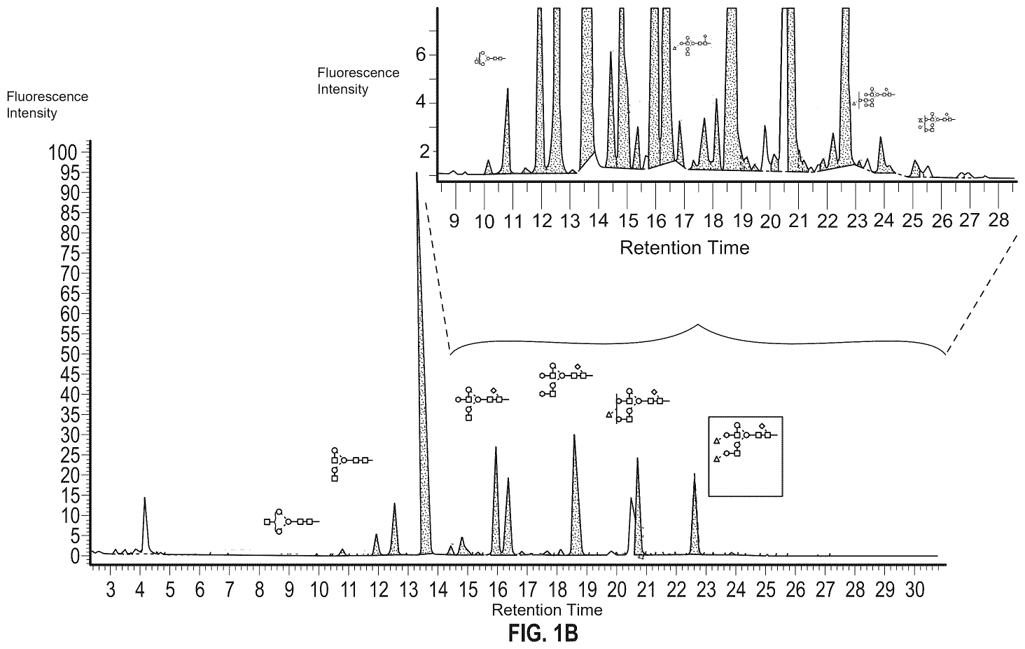

Elimination of the need for a mass spectrometer dramatically reduces the complexity of the instrumentation needed for a glycan assay and thus eliminates a barrier to its widespread usage. Typical released glycan panels are shown in FIG. 1A for the NIST monoclonal antibody and in FIG. 1B for an antibody with dual N-linked glycosylation sites. From these chromatograms, each antibody can be seen to be comprised of a 5-10 highly abundant glycoforms and dozens of much lower abundance glycoforms. In the case of the antibody with dual glycosylation sites (FIG. 1B), the number of low abundance glycoforms is much higher and many of these species co-elute with one another. In the absence of mass confirmation capabilities, the confidence of identification of these species based on retention time alone is extremely low.

In one embodiment, the chromatographic complexity of a released glycan separation is reduced through prefractionation using lectins. Lectins are class of proteins that naturally exhibit specificity towards different glycoforms and structures. Prefractionation of released glycans is achieved by passing the released glycans through a device in which one or more lectin proteins are coupled to a structured array. In this manner, the released glycan sample can be prefractionated prior to optical only detection.

Released glycan sample preparation is comprised of five stages: enzymatic cleavage of the glycan from the protein/polypeptide; separation of the protein/polypeptide components from the glycan components; tagging of the newly freed glycosylamine with an NHS carbamate group connected to a mass and fluorescent tag; separation of the untagged material from the tagged glycans; and analysis by LC-FLR-MS. Also included in the sample preparation process is an initial separation of the target protein/polypeptide from the background host cell protein material; this is typically achieved with an affinity separation. Herein a device is described that leverages combined affinity purification and enzymatic release of the glycans, and combined glycan fractionation based on a glycan affinity panel to produce samples suitable for analysis by LC.

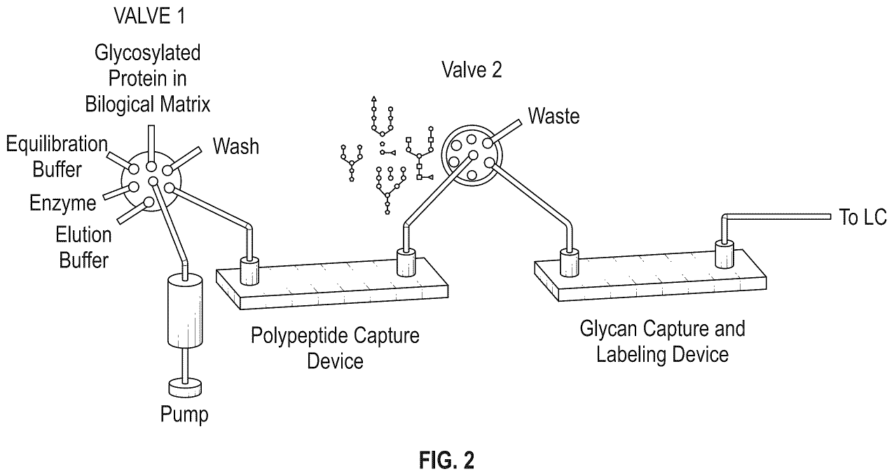

A system for isolating and tagging a glycan is shown in FIG. 2. The system includes a polypeptide capture device and a glycan capture and labeling device. As shown in FIG. 2, the polypeptide capture device is fluidically coupled to the glycan capture and labeling device. Specifically, the outlet of the polypeptide capture device is fluidically coupled to the inlet of the glycan capture and labeling device. One or more tubes or valves may form a fluidic passageway between the polypeptide capture device and the glycan capture and labeling device.

The sample preparation process includes an initial separation of the target polypeptide (e.g., a protein) from the background host cell material using affinity separation. In the system depicted in FIG. 2, the polypeptide capture device includes an affinity capture agent. The affinity capture agent is an agent that can selectively bind to a protein/polypeptide of interest. In a preferred embodiment, the affinity capture agent will bind to a broad class of proteins/polypeptides. Specifically, the affinity capture agent will bind to many different polypeptides. For example, Protein A, Protein G, Protein A/G, and Protein L are proteins that bind to most species and subclasses of IgG antibodies. In alternate embodiments, an antigen for the antibody being captured can be used as an affinity capture agent. In another embodiment, affinity to a scaffold system such as a monobody can be developed via evolutionary display approaches. Other types of affinity capture agents, for use in the polypeptide capture device include platform affinity agents that can be optimized against a given target or target class of protein/polypeptides, such as DARPins, affibodies, repebodies, fynomers, anticalins, His-tags, and aptamers.

The affinity capture agent is, in one embodiment, immobilized to an interior surface of the polypeptide capture device. The polypeptide capture device includes an inlet, an interior chamber, and an outlet. The affinity capture agent is immobilized to a surface of the interior chamber. The affinity capture agent can be immobilized to the surface through physical interactions, covalent bonding, or bioaffinity. In a preferred embodiment, the affinity agent is covalently coupled to the surface of the interior chamber. Covalent coupling of the protein-based affinity agents to the surface can be accomplished through direct reaction of one or more amino acid side chains of the protein-based affinity agent with the surface. Amino acids having hydroxyl side groups (e.g., serine) or amine terminal side groups (e.g., lysine) are used to covalently link the protein-based affinity agent to the surface. Alternatively, a linking group may be coupled to the surface of the interior chamber. The linking group can be a bifunctional group that can form covalent bonds to both the surface and the affinity capture agent.

In another embodiment, the chamber of the polypeptide capture device may enclose resin particles with one or more affinity capture agents bonded to the resin. For example, the resin can be formed from cross-linked agarose beads with the affinity capture agent (e.g., Protein A) covalently bound to the resin. The affinity capture agent can be bonded to the resin through the use of reductive amination coupling strategies. The particles may be dispersed within the interior chamber of the polypeptide capture device. The inlet and outlet of the chamber may include one or more frits that retain the resin particles within the chamber. In an alternate embodiment, nanoparticles can be dispersed on an interior surface of the polypeptide capture device. The nanoparticles are selected from a material (metal, polymers, silica/glass, etc.) that captures the polypeptides of interest as the sample passes through the polypeptide capture device.

During use, a sample is passed into the polypeptide capture device through the inlet and into the interior chamber. The sample can be unclarified or clarified biological material. The sample may be a blood sample or may be a sample taken from a bioreactor. The sample is passed into and through the chamber where the sample contacts the affinity capture agent. Antibodies and other glycosylated polypeptides present in the sample can be captured by the affinity capture agent as the sample passes through the chamber. In some embodiments the flow rate of the sample through the chamber may be adjusted to allow sufficient time for the proteins/polypeptides or antibodies in the sample to bind to the affinity agent. In other embodiments, ridged structures may be used to facilitate mixing and binding of material.

One or more valves may be used to introduce fluids into the polypeptide capture device. For example, a multi-port valve may be coupled to the inlet of the polypeptide capture device. The multiport valve may include multiple input ports and output ports. An output port of the valve can be coupled to the inlet of the polypeptide capture device to allow the selective introduction of fluids into the polypeptide capture device. The multiport valve can also include a plurality of input ports. In one embodiment, the input ports of the multiport valve can be coupled to one or more of: an equilibrium buffer; enzyme solution (e.g., a glycanase enzyme solution); elution buffer; sample introduction (e.g., a glycosylated protein/polypeptide in a biological sample); and a wash solution.

The first step of the process of capturing glycans released from a glycoprotein/glycopeptide is introducing the sample into the polypeptide capture device. The solvent or sample is chosen to promote binding of the glycoprotein/glycopeptide being studied with the affinity capture agent. As shown in FIG. 2, the sample travels through the first valve and into the polypeptide capture device. A pump, connected to the valve, may provide pressure forcing the sample through the valve into the polypeptide capture device. In an alternate method, centrifugal force may be applied to the polypeptide capture device, after the sample is introduced into the polypeptide capture device, to provide positive pressure on the sample, pushing the sample past the affinity capture agent.

The sample can be dissolved or mixed in a solvent that promotes binding of the glycoprotein/glycopeptide of interest. Some parameters that can promote binding of the glycoprotein/glycopeptide to the affinity capture agent include salt concentration and buffer pH. In some embodiments, it is preferred to have a salt concentration of <50 mM, preferably <5 mM. Preferably the pH of the sample is kept to less than 8. In the absence of structures designed to facilitate mixing of fluidic components, the flow rate of the sample through the polypeptide capture device is determined, in part, by the kinetics of release of the affinity capture agent and captured protein/polypeptide. To ensure capture of the desired proteins/polypeptides in the sample, the flow rate of the sample is reduced to allow a residence time of less than 50% of the kinetic release half-life. For example, Protein A has a kinetic release time (half-life) of about 50 minutes for most proteins/polypeptides. To ensure sufficient capture of proteins/polypeptides by Protein A, the residence time of the sample in contact with the affinity capture agents should be set to about 25 minutes or less.

Flow rates of 0.1 μL/sec to 1.0 mL/sec can be used depending on the affinity capture agent and the configuration of the polypeptide capture device. In the absence of any specific structures in the polypeptide capture device that provide mixing and increase the effective surface area of the interior surface, the flow rates will be relatively slow to ensure enough time for diffusion of the proteins of interest to the affinity capture agents (e.g., 0.1 μL/sec to 10 μL/sec). Using structures that facilitate mixing and/or the flow path through the polypeptide capture device can allow faster flow rates (e.g., from 0.1 mL/sec to 1.0 mL/sec)

A second valve (Valve 2 in FIG. 2) can be used to control where the fluid flows from the outlet of the polypeptide capture device. In an embodiment, Valve 2 has at least one input and two outputs. Valve 2 can be a three-way valve or a multiport valve depending on the output and inputs needed for the particular system. In the embodiment depicted in FIG. 2, the outlet of the polypeptide capture device is coupled to Valve 2. Valve 2 can have two outlet positions. The first outlet position of Valve 2 transfers the fluid exiting the polypeptide capture device to a waste container. During use, when a sample or wash fluid is being flowed through the polypeptide capture device, the fluid is usually transferred to a waste container. The second outlet position of Valve 2 leads to the input of the glycan capture and labeling device. Valve 2 is placed in the second outlet position when fluids from the polypeptide capture device are being transferred to the glycan capture and labeling device.

After the proteins are bound to the affinity capture agent, one or more optional washes may be performed to assist in removal of host cell proteins and other interferents. In one embodiment, a series of washes utilizing different pH buffers are used to remove unwanted material from the polypeptide capture device. For example, a “high pH” buffer wash can be used, where the pH of the buffer solution is greater than 7, greater than 8, greater than 9, or greater than 10. In some embodiments, a series of high pH buffer solutions can be used to wash the polypeptide capture device with the pH being increased or decreased with each sequential wash between pH 7 and pH 10, or greater.

Another wash that can be performed uses a detergent to remove unwanted material from the polypeptide capture device. Exemplary detergents include, but are not limited to, 1 M urea+isopropyl alcohol, tween 80, and polysorbate. The use of detergent can be the only wash step or can be combined with high pH wash steps.

After the desired proteins/polypeptides are bound to the affinity capture agent, the glycans are cleaved from the glycosylated proteins/polypeptides. The process of cleaving the glycans from the bound proteins/polypeptides is achieved, in one embodiment, by passing a solution comprising a glycanase through the polypeptide capture device. A glycanase is an enzyme that cleaves glycans from glycosylated proteins/polypeptides. In one embodiment, the glycanase is an N-glycanase. An N-glycanase cleaves N-linked glycans from glycosylated proteins/polypeptides. The N-glycanase cleaves the glycans from the glycosylated protein/polypeptide by hydrolyzing the amide bond between the innermost glucose unit and the side chain of the amino acid that is bonded to the glycan. Exemplary glycanases include, but are not limited to, PNGaseF, PNGaseA, Endoglycosidase H, and Endoglycosidase F.

Cleavage of the glycans from the bound proteins/polypeptides can be achieved using a flow through process. In this process, a solution of the glycanase is flowed through the polypeptide capture device. The glycanase releases the glycans from the bound peptides/polypeptides and the released glycans are carried with the flowing fluid out of the polypeptide capture device. The use of a flow-through enzymatic digestion strategy allows easy removal of the glycans, without the need to remove the proteins/polypeptides. Amine free buffers are needed for the labeling step when using N-Hydroxysuccinimidyl (NHS) carbamate coupling of the label to the glycan. However, elution buffers for removal of proteins/polypeptides from affinity columns often utilize amine containing buffer salts. Typical processes that rely on elution of a protein/polypeptide from an affinity column, prior to glycan removal and analysis, are faced with the problem of removing or changing the buffer after the proteins/polypeptide have been eluted. The claimed flow through process, therefore, provides the benefit of cleanly separating the glycans from the base proteins/polypeptides in an amine-free buffer allowing facile labeling of the glycan in subsequent processing steps. A second benefit in the claimed process is the elimination of an acid neutralization step that is typically required following low pH elution of protein from the polypeptide capture step.

Once cleaved, the glycans are fluidically passed to the glycan capture and labeling device. In the embodiment shown in FIG. 2, Valve 2 is set to the second position, which leads to the input of the glycan capture and labeling device.

After removal of the glycans from the polypeptide capture device, the polypeptide capture device is prepared for capture of a sample by removal of the bound proteins/polypeptides. The deglycosylated proteins/polypeptides can be removed by elution with an appropriate elution solution comprising an elution agent. In one embodiment, the elution solution is an acidic solution comprising an elution agent. The acidic elution solution has a pH of less than 7, less than 5, or less than 3. Typical elution solutions used to clean out the deglycosylated proteins/polypeptides have a pH from 2.0 to 3.2. The concentration of the elution agent is about 50 mM to about 150 mM. In one embodiment, the elution agent is citric acid. Other elution agents include, but are not limited to, aqueous solutions comprising phosphoric acid, hydrochloric acid, or sulfuric acid.

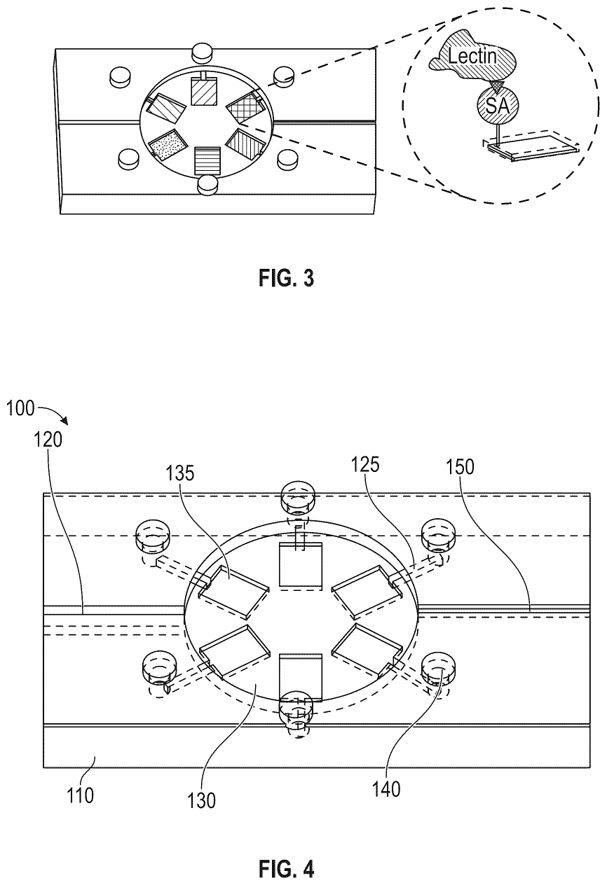

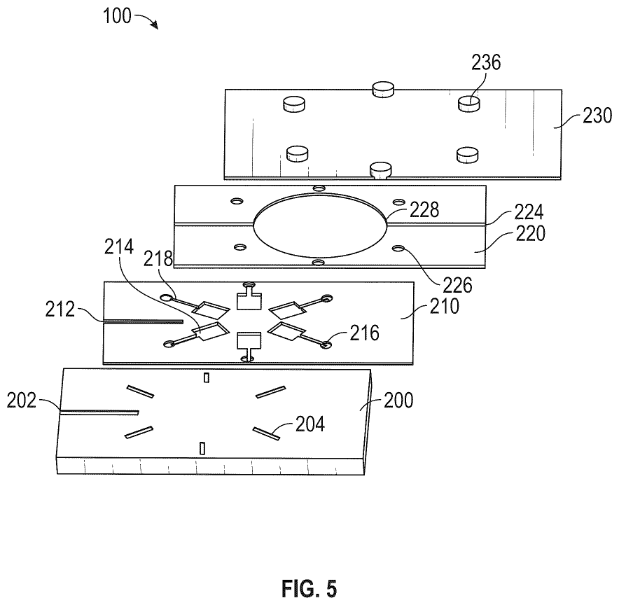

FIG. 3 depicts a top view of a glycan labeling and capture device. The glycan labeling and capture device includes one or more wells disposed in a reaction chamber; each well comprising one or more glycan affinity capture agents coupled to an interior surface of the well. In a preferred embodiment, the one or more glycan affinity agents are lectin proteins. FIG. 4 shows a projection view of the glycan capture and labeling device 100. FIG. 5 shows an exploded view of the glycan capture and labeling device 100. Glycan capture and labeling device 100 includes a body 110 having an inlet 120, a reaction chamber 130, reagent reservoirs 140, and an outlet 150. Reaction chamber 130 includes a plurality of wells 135. Each of the plurality of wells 135 is fluidically coupled to one of the plurality of reagent reservoirs 140 as depicted in the figures. Reservoir conduits 125 fluidically coupled the reagent reservoirs to the wells 135. Reaction chamber 130 is an open chamber that allows fluid from inlet 120 to enter wells 135 disposed in the reaction chamber. The fluid in reaction chamber 130 will flow out of the reaction chamber through outlet 150. The fluid reaction chamber and wells can be made from materials capable of binding to the affinity agent directly, or indirectly through a linker. Suitable materials include, but are not limited to, cyclic olefin polymers (COP), cyclic olefin copolymer (COC), and epoxy resins. In other embodiments, the glycan capture and labeling device body is made from silica/glass or metal (e.g., steel or titanium) and a layer of reactive material is applied to the body to allow coupling of the glycan affinity capture agents. For example, a layer of COP, COC, or epoxy resin can be applied to the body to allow coupling of the glycan capture agents.

FIG. 5 shows one embodiment of the construction of the glycan capture and labeling device 100. In this embodiment, the device is constructed of multiple layers connected to each other. In the embodiment shown in FIG. 5, the device includes a base layer 200, a well layer 210, a reaction chamber layer 220, and a reservoir layer 230. The layers can be connected by an adhesive that is resistant to the fluids used during testing (predominantly water). The adhesive forms a seal keeping the fluids within the structures formed by the layers. The layers can be formed by etching the depicted passageways and openings into a material. Alternatively, one or more of the layers can be formed by chemical etching, laser ablation, and injection molding, a 3D printing or additive manufacturing process. In some embodiments, the layers are made individually and then adhered together. In certain embodiments, the layers of the device are built sequentially, stacked one on top of another. Sealing of the layers can be done either thermally, using an adhesive, or by diffusion bonding of the layers.

The base layer 200 includes a base inlet passageway 202 and reservoir conduits 204 formed in a body.

The well layer 210 includes a well inlet passageway 212. The well layer also includes a plurality of openings 214 which define wells 135 in the glycan capture and labeling device. The sidewalls of the openings define the boundaries of the wells, while base layer 200 defines the bottom of the wells, when the well layer is attached to the base layer. The well layer further includes reagent reservoir base 216 and reservoir upper conduits 218. The reservoir lower conduit 208 and the reservoir upper conduit 218 combine to form the reservoir conduit 125 (depicted in FIG. 4) when the well layer is attached to the base layer.

The reaction chamber layer 220 includes reaction chamber inlet passageway 222, reaction chamber outlet passageway 224, and reservoir compartments 226. The reaction chamber layer also includes a reaction chamber opening 228 which defines the sidewalls of the reaction chamber of the glycan capture and labeling device. Base inlet passageway 202, well inlet passageway 212, and reaction chamber inlet passageway 222 combine to form inlet 120 (depicted in FIG. 4), when the layers are attached to each other. The reaction chamber outlet passageway 224 defines the glycan capture and labeling device outlet 150. The reaction chamber opening 228 defines the outer sidewalls of the reaction chamber when the layers are attached to each other.

The reservoir layer 230 includes a plurality of reservoirs 236. Reservoirs 236 are aligned with reservoir compartments 226 and reservoir bases 216 when the layers are attached to each other. Reservoirs 236 hold different dyes that are used to tag the glycans captured in the wells. In an embodiment, reservoirs 236 can be removable so that the dyes can be replaced or changed. Reservoirs 236 can be sealed to contain the dyes. In one embodiment, upon application of pressure to a reservoir, an opening can form in the reservoir allowing the dye from the reservoir to enter the reservoir compartment and base. The pressure from opening the reservoir pushes the dye into the wells where the dye reacts with glycans.

Each of the wells 135 include glycan affinity capture agents. In an embodiment, each well 135 includes a different glycan affinity capture agent specific for different classes of glycans. Exemplary glycan affinity capture agents include lectin proteins and scaffolds engineered to have specific binding behavior towards certain subclasses of glycans. Glycan specific scaffolds can be formed from DARPins, affibodies, repebodies, fynomers, anticalins, and aptamers.

In an embodiment, lectin proteins can be used as glycan affinity capture agents. Lectin proteins have specific selectivity towards glycans. Each different type of lectin protein binds to a specific class of glycans based on the structure of the glycan. The lectin proteins bind to specific classes of glycans among a mixture of glycans. Additionally, lectin proteins have a multivalency that leads to high-affinity binding to the glycans. By selecting specific lectin proteins, a plurality of glycans can be isolated in the wells within the glycan capture and labeling device, with each well containing a specific class of glycans.

Lectin proteins that can be used in the glycan capture and labeling device can be selected for specific glycans of interest. Glycan binding lectin proteins can be classified according to the type of carbohydrate structure that the lectin exhibits specificity.

Mannose binding lectins include, but are not limited to, Arum maculatum agglutinin (AMA), Concanavalin-A (ConA), Galanthus nivalis lectin (GNA, GNL), Hippeastrum hybrid lectin (HHA, HHL), Morniga M agglutinin (MNA-M), Narcissus pseudonarcissus lectin (NPA), Sambucus nigra agglutinin II (SNA-II), and Urtica dioica lectin (UDA) and Lens culinaris hemagglutinin (LcH).

Complex N-glycan binding lectins include, but are not limited to, Agaricus bisporus (ABA, ABL), Colchicum autumnale (CA), Caragana arborescens (CAA), Tulipa lectin (TL), Datura stramonium (DSA), Phaseolus vulgaris-E (PHA-E), Phaseolus vulgaris-L (PHA-L), and Robinia Pseudoacacia (RPA).

O-Glycan binding lectins include, but are not limited to, Amaranthus caudatus (ACA, ACL), peanut agglutinin (PNA), Artocarpus integrifolia (AIA, Jacalin), Codium fragile (CF), Maclura pomifera (MPA, MPL), Helix pomatia agglutinin (HPA), and Helix aspersa agglutinin (HAA) (FIG. 5).

Fucose binding lectins include, but are not limited to, Aleuria aurantia lectin (AAL), Aspergillus oryzae lectin (AOL), Laburnum alpinum lectin (LAA), Lens culinaris hemagglutinin (LcH, LcA), Lotus tetragonolobus lectin (LTL), Pisum sativum agglutinin (PSA), Psophocarpus tetragonolobus lectin-I (PTL/PTA I) and -II (PTL/PTA II), Trichosanthes japonica agglutinin II (TJA-II), and Ulex europaeus agglutinin-I (UEA-I).

Sialic acid binding lectins include, but are not limited to, cholera toxin B (CTB), Maackia amurensis-I (MAL-I, MAM, MAL), Maackia amurensis-II (MAL-II, MAH), Polyporus squamosus (PSL), Trichosanthes japonica-I (TJA-I), and Sambucus nigra-I (SNA-I).

Terminal GlcNAc and chitin binding lectins include, but are not limited to, Griffonia simplicifolia-II (GS-II), Phytolacca Americana (PWA), Ulex Europaeus-II (UEA-II), and wheat germ agglutinin (WGA).

Terminal galactose and LacNAc binding lectins include, but are not limited to, Bauhinia purpurea lectin (BPA, BPL), Erythrina cristagalli agglutinin (ECA, ECL), Grif fonia simplicifolia-I (GS-I), Lycopersicon esculentum agglutinin (LEA), Marasmius oreades agglutinin (MOA), Pseudomonas aeruginosa-IL (PA-IL), Ricinus communis agglutinin (RCA-I, RCA120), Sophora japonica agglutinin (SJA), and Solanum tuberosum lectin (STA, STL).

Terminal GalNAc binding lectins include, but are not limited to, Cytisus scoparius Lectin (CSA), Dolichos biflorus (DBA), soybean agglutinin (SBA), Vicia villosa lectin (VVL, VVA), and Wisteria floribunda agglutinin (WFA, WFL).

In one embodiment, the glycan capture device includes a plurality of wells. Each of the plurality of wells includes a different glycan affinity capture agent. In an embodiment, each well can include a different lectin protein selected from one of the lectin types described above. For example, one of the wells can include mannose binding lectins and another of the wells can include fucose binding lectins. Additional wells can include additional types of lectin proteins. In a preferred embodiment, each of the wells includes a lectin protein from a different class of lectin proteins as described above. In another embodiment, affinity agents derived from monobody or other scaffold designs may be developed with specificity to specific glycans and may be used as the glycan affinity agent.

The glycan affinity capture agents are coupled to the interior surface of the wells through a coupling reagent. Alternatively, the glycan affinity capture agents can be coupled to the interior surface of the wells directly through binding of a functional group of the glycan affinity capture agents. For lectin proteins, streptavidin can be used to bind the lectin proteins to the surface. Streptavidin can be bound to the interior surface of the wells. Biotinylated lectin proteins bind to the streptavidin such that the lectin proteins are substantially immobilized to the surface. Biotinylated-streptavidin coupling of lectins to the surface allows a generic immobilization scheme to be used. Any lectin protein can be biotinylated such that when the biotinylated lectin protein is introduced into the wells it becomes bound to the streptavidin. In this manner a single immobilization strategy can be used for a plurality of different lectins.

The glycan capture is performed by passing the solution of glycans produced in the polypeptide capture device directly into the reaction chamber of the glycan capture and labeling device. As the solution passes into and through the reaction chamber, glycans corresponding to the specific glycan affinity capture agent(s) disposed in the well(s) are trapped through affinity binding to the glycan affinity capture agents. As depicted in FIG. 3, the glycan capture and labeling device includes an inlet and an outlet. The inlet is fluidically coupled to the outlet of the polypeptide capture device as shown in FIG. 2. The outlet is fluidically coupled to an analytical system (e.g., a liquid chromatography system). A valve (not shown) may be present between the outlet and the analytical system to allow the fluid flowing from the glycan capture and labeling device to be sent to a waste container or directly to the analytical device, depending on the processing step being performed.

After the glycan containing solution passes through the reaction chamber, one or more wash steps are performed to remove any unbound glycans. The wash steps are performed with amine free buffer (e.g., sodium bicarbonate) to remove unbound glycans. After the wash steps are completed, air is passed through the reaction chamber to remove all remaining liquid.

After the glycans have been captured, cleaned, and dried, the glycans can be released and transferred to an analytical device. Alternatively, the glycans can be labeled with a suitable tag that will improve the ability to discern the different glycans in the analytical device, prior to release and transfer of the glycans to the analytical device.

In one embodiment, the glycan capture and labeling device includes one or more reagent reservoirs comprising a dye that can bind to a glycan, as shown in FIG. 3. Each reagent reservoir is fluidically coupled to a single well. After the glycans are captured, each of the reagent reservoirs are activated such that the dye in each reservoir is transferred to the well fluidically coupled to the reservoir. In one embodiment, the fluid may be transferred though the use of a pump coupled to the reagent reservoir. The pump can be activated to transfer fluid from the reagent reservoir into the well. Alternatively, the reagent reservoir may be a pressurized reservoir. Application of pressure to the pressurized reservoir can force the dye into the well that is fluidically coupled to the reservoir.

In one embodiment, the reagent reservoir relies on hydrophilic interactions between the dye containing liquid and the cap of the device to contain the dye within each well. To inhibit bleeding and spreading between wells, the captured glycans are air-dried before being treated with the dyes in the reservoirs, as discussed above.

The glycans in the well are incubated with the dye for a time sufficient to allow the dye to bind to the glycans in the well. Labeling of glycans with a dye is beneficial to detecting glycans because it improves both sensitivity of the detection and selectivity by altering the chromatographic behavior of the glycans. Strategies and methods for labeling glycans are described in U.S. Pat. Nos. 8,124,792 and 11,352,325 and U.S. Patent Application Publication Nos. U.S. 2018/0094293; U.S. 2019/0331669; and U.S. 2020/0332028, all of which are incorporated herein by reference.

Dyes can be selectively attached to specific carbohydrate groups of the glycan. In one embodiment, a dye is coupled to one or more of the terminal carbohydrate groups of the glycans. Dyes can be selectively coupled to the terminal carbohydrate of the glycans through enzyme mediated reactions. For example, an enzymatic catalyzed tagging of a glycan can be tailored to only tag a specific terminal carbohydrate. In one embodiment, dyes can specifically target fucose, sialic acid, high mannose and terminal GlcNAc carbohydrates. In some embodiments, the glycans bound to the glycan affinity capture agent will have different terminal carbohydrates. The specificity of the dye can therefore add additional prefractionation of the glycans by only tagging some, but not all of the glycans captured within a well.

After the glycans are labeled, the labeled glycans are washed with a buffer to remove excess and unreacted dye from the wells and reaction chamber. After washing of the labeled glycans is complete, the glycans are removed from the glycan affinity capture agents and passed to the analytical device (e.g., a liquid chromatography system). The glycans are removed by the applying a carbohydrate solution to the reaction chamber and wells. The carbohydrates in the solution replace the glycans bound to the glycan affinity capture agents. The released labeled glycans are transferred out of the reaction chamber through the outlet of the glycan capture and labeling device. The outlet of the glycan capture and labeling device is coupled to the analytical device such that the glycans can be directly transferred to the analytical device as they are removed from the wells.

In one embodiment, the dye is a fluorescent compound. The use of different fluorescent labels for each well of the glycan can provide enhanced detection of the different glycan species. For example, fluorescent labels used in each well can have different fluorescence, this allows monitoring of different classes of glycan using different wavelengths. In this embodiment, the glycans can be monitored over a plurality of fluorescent channels corresponding to the fluorescent labels used to tag the glycans. The number of channels monitored will correspond to the number of pre-fractionated and differentially labeled glycan subtypes.

Additionally, the dye coupled to the glycan can alter the chromatographic behavior (e.g., the retention time) of the glycans, depending on the analytical method. The change in retention time can help separate glycans that typically have similar elution rates.

In one example, rhodamine labeling is used to label high mannose type glycans and fluorescein labeling is used for labeling N-linked glycans containing a core fucose. The two subclasses (rhodamine and fluorescein labeled glycans) would experience differential chromatographic retention during the HILIC separation as well as differential excitation-emission transitions, thus providing two parallel dimensions of separation. The differential retention as a function of glycan subclass serves to reduce the local complexity of the reduced glycan panel; combined with the multichannel nature of the detection, this scheme affords acceleration of the gradient and reduces the required assay time.

### EXAMPLE

### Forming Lectin-Affixed Polystyrene Beads

Carboxlate polystyrene beads were coupled to (5S)-N-(5-Amino-1-carboxypentyl) iminodiacetic acid (NTA-NH2) using standard EDAC chemistry. NTA coupled beads were washed and incubated in 100 mM NiCl₂ for 1 hour to occupy the nitrilotriacetic acid moiety. His-tagged lectin was added in 5× molar excess to the particles. The lectin-affixed beads were washed 3× with PBS and subsequently incubated in a mixture containing 2 mM calcium acetate, 2 mM magnesium acetate, and 2 mM manganese acetate for 1 hour. The lectin-affixed beads were subsequently washed 4× with PBS to remove excess salt ions.

### Lectin Specificity of High Mannose Glycans Using Lectin-Affixed Polystyrene Beads

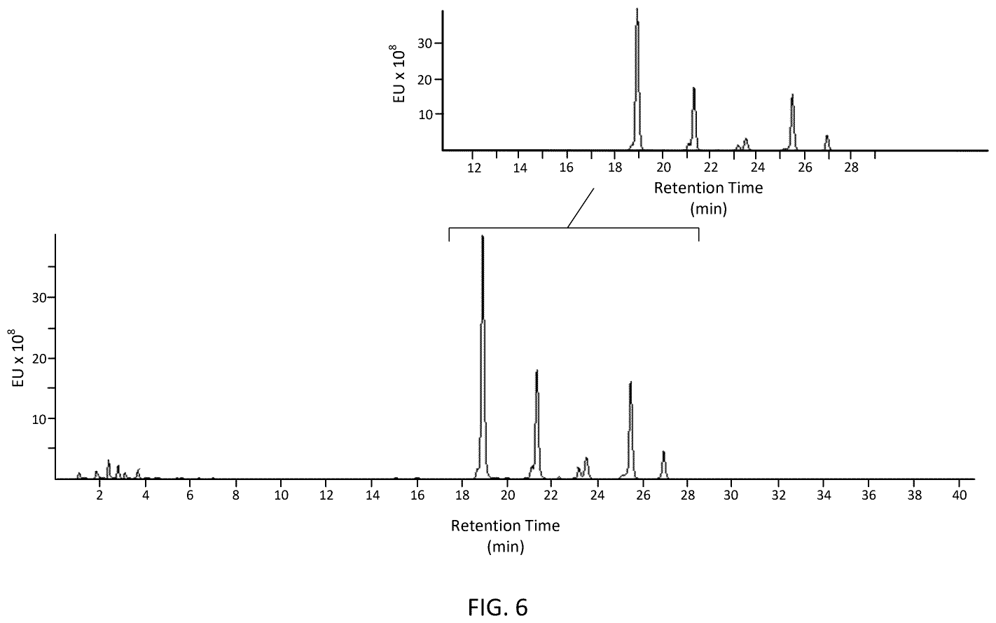

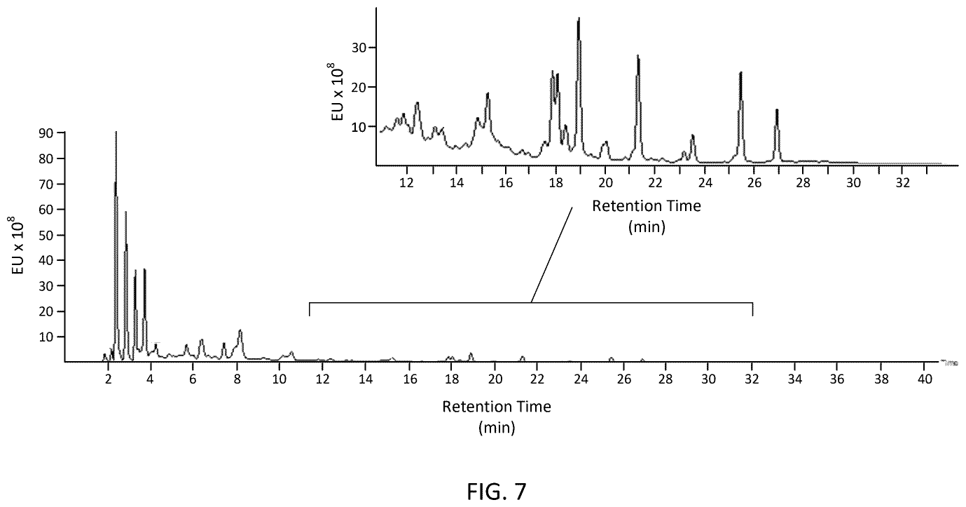

Lectin specificity for high mannose glycans was tested using the lectin-affixed beads. A heterogeneous mixture of high mannose glycans was prepared from stock protein via glycolysis using PNGase F and subsequent tagging using the Rapifluor-MS tag. The resultant slurry contained a mixture of tagged glycans, residual glycans, unreacted tag, deglycosylated protein, and deglycosylated and variably tagged protein. The slurry was incubated with a 50 μL aliquot of the lectin-affixed polystyrene beads for 20 minutes. The liquid containing the unbound components was removed, and the beads were washed 4 times with PBS. The glycans were eluted from the lectin-affixed beads using a 50 μL volume of 1 M alpha-methyl mannose that was incubated with the beads for 30 minutes. The resulting supernatant was analyzed by HILIC-FLR, yielding the chromatogram in FIG. 6. The liquid containing the unbound material after reaction of the initial slurry with the lectin-affixed polystyrene beads was also analyzed by HILIC-FLR, and is shown in FIG. 7.

FIG. 6 shows enrichment of high mannose glycoforms following lectin affinity binding and elution. FIG. 7 shows the unbound material from the lectin affinity binding step of this treatment. Comparing FIG. 6 to FIG. 7, it can be seen that sample obtained from elution of the bound glycans from the lectin affixed polystyrene beads provides a sample that has significantly reduced amounts of undesired compounds.

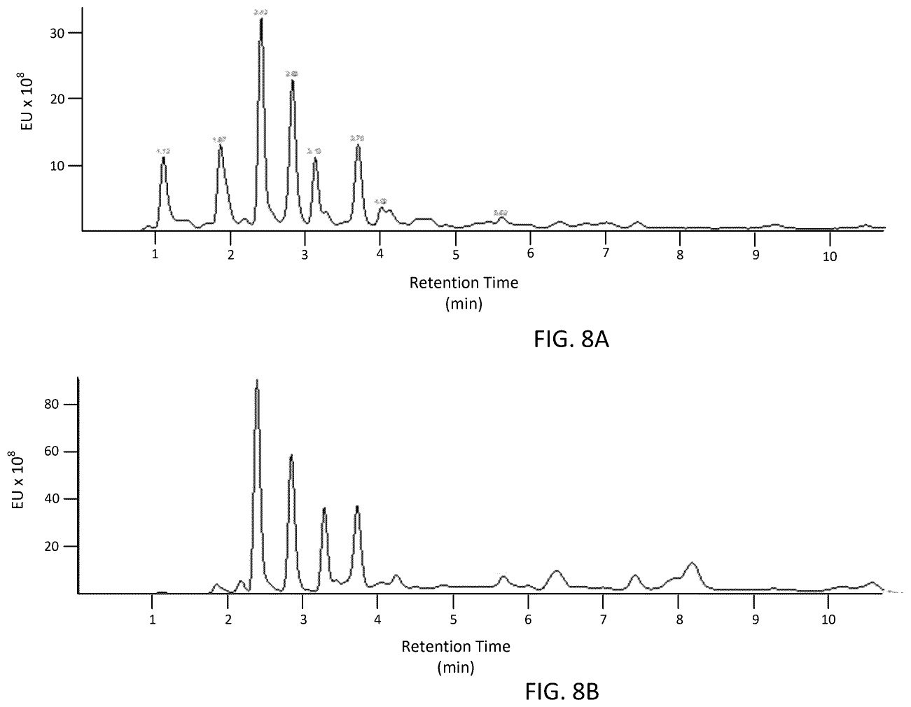

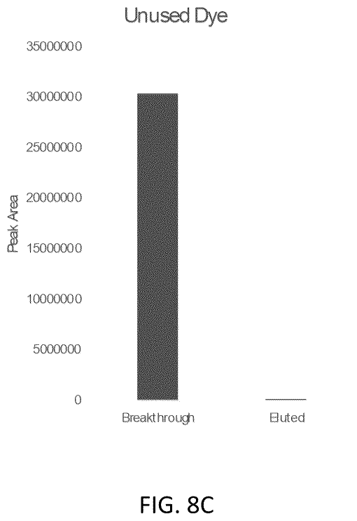

FIGS. 8A-8C depict residual dye peaks as separated and measured by HILIC-FLR and illustrate the magnitude of removal of the excess dye. FIGS. 8A and 8B highlight the region of the chromatogram where reacted dye and protein elute (0 to 11 minutes). The signal of the dye species in this region are compared for the elution fraction (FIG. 8A) and the unbound fraction (FIG. 8B). FIG. 8C shows the quantitation of the dye peaks in FIG. 8A and FIG. 8B, demonstrating a greater than 200 fold reduction in unreacted dye and tagged protein.

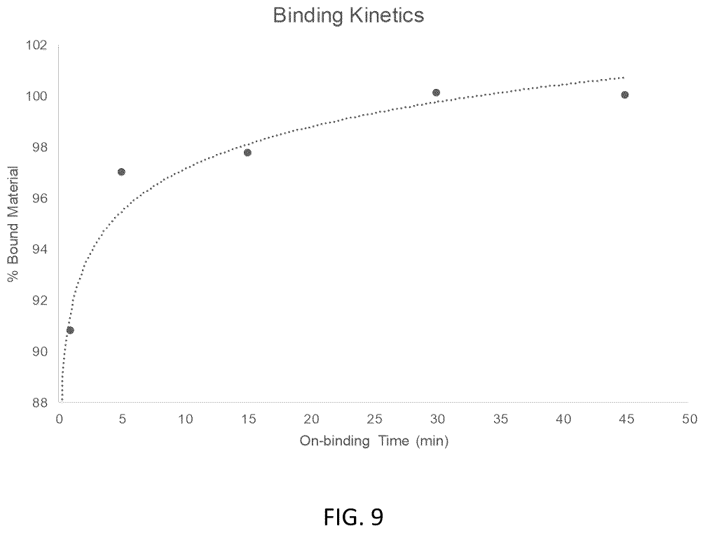

Using the same preparation as described above, on-binding kinetics were studied using variable times for the incubation of the sugars with the lectin-affixed beads. The kinetic curve shown in FIG. 9 demonstrates the rapid binding of the sugars to the protein, even with the fluorescent tag attached. This kinetics binding experiment demonstrates that binding is complete within ˜5 minutes.

Further modifications and alternative embodiments of various aspects of the technology will be apparent to those skilled in the art in view of this description. Accordingly, this description is to be construed as illustrative only and is for the purpose of teaching those skilled in the art the general manner of carrying out the present technology. It is to be understood that the forms of the present technology shown and described herein are to be taken as examples of embodiments. Elements and materials may be substituted for those illustrated and described herein, parts and processes may be reversed, and certain features of the technology may be utilized independently, all as would be apparent to one skilled in the art after having the benefit of this description of the technology. Changes may be made in the elements described herein without departing from the spirit and scope of the technology as described in the following claims.

1. A method of capturing glycans released from a protein or polypeptide, the method comprising:
passing the protein or polypeptide through a polypeptide capture device comprising an affinity capture agent, wherein at least a portion of the protein or polypeptide passing through the polypeptide capture device is captured to the affinity capture agent;
treating the captured protein or polypeptide with a glycanase such that one or more glycans are removed from the captured protein or polypeptide; and
passing the released glycans into a glycan capture and labeling device, wherein the glycan capture and labeling device comprises one or more glycan affinity capture agents, wherein the one or more glycan affinity capture agents have an affinity for a specific structural feature of a glycan.

2. The method of claim 1, wherein the affinity capture agent comprises one or more proteins selected from the group consisting of Protein A, Protein G, Protein A/F, and Protein L.

3. The method of claim 1, wherein the affinity capture agent comprises antibodies or nanoparticles that bind to the protein or polypeptide.

4. The method of claim 1, wherein the glycanase is PNGaseF, PNGaseA, Endoglycosidase H, or Endoglycosidase F.

5. The method of claim 1, wherein the polypeptide capture device is fluidically coupled to the glycan capture and labeling device.

6. The method of claim 1, wherein the glycans removed from the captured protein are fluidically transferred from the polypeptide capture device to the glycan capture and labeling device.

7. The method of claim 1, wherein the glycan affinity capture agent is a lectin protein.

8. The method of claim 7, wherein the lectin protein is selected from the group consisting of mannose binding lectins, complex N-glycan binding lectins, O-glycan binding lectins, fucose binding lectins, sialic acid binding lectins, terminal GlcNAc and chitin binding lectins, terminal galactose and LacNAc binding lectins, and terminal GalNAc binding lectins.

9. The method of claim 7, wherein the lectin proteins include one or more lectin proteins selected from the group consisting of mannose binding lectins, complex N-glycan binding lectins, O-glycan binding lectins, fucose binding lectins, sialic acid binding lectins, terminal GlcNAc binding lectins, chitin binding lectins, terminal galactose binding lectins, LacNAc binding lectins, and terminal GalNAc binding lectins.

10. The method of claim 1, further comprising removing the protein or polypeptide from the polypeptide capture device.

11. The method of claim 1, further comprising incubating each glycan bound onto the glycan affinity capture agent with a dye that bonds to the glycan.

12. The method of claim 11, wherein a different dye is applied to glycans bound to each of the different glycan affinity capture agents.

13. The method of claim 11, further comprising eluting the glycans from the glycan capture and labeling device.

14.-25. (canceled)
